(19) United States
(12) Patent Application Publication
(10) Pub. No.: US 2014/0349306 A1
(43) Pub. Date: Nov. 27, 2014

(54) NOVEL HUMAN EXOSOMAL PROTEINS AND USE THEREOF

(71) Applicant: KONKUK UNIVERSITY INDUSTRIAL COOPERATION CORP., Seoul (KR)

(72) Inventors: Hyewon CHUNG, Seoul (KR); Hyung-Soon PARK, Seoul (KR); Hyun-Jung LIM, Seoul (KR)

(73) Assignee: KONKUK UNIVERSITY INDUSTRIAL COOPERATION CORP., Seoul (KR)

(21) Appl. No.: 14/096,682

(22) Filed: Dec. 4, 2013

(30) Foreign Application Priority Data
May 21, 2013 (KR) 10-2013-0057331

(51) Int. Cl. G01N 33/573

(52) U.S. Cl. CPC G01N 33/573
USPC 435/7.1

(57) ABSTRACT

There are provided novel human exosomal proteins and use thereof.

### CROSS-REFERENCE TO RELATED APPLICATION(S)

This application claims the priority of Korean Patent Application No. 10-2013-0057331 filed on May 21, 2013, the entire contents of which is incorporated herein by reference.

### BACKGROUND OF THE INVENTION

1. Field of the Invention

The present invention relates to novel human exosomal proteins, and a use thereof.

2. Description of the Related Art

In general, exosome is a microvesicle originated from endosome, and a round vesicle having a diameter of 30 to 100 nm. For exosome, multivescicular bodies in most cells are fused to secrete exosome outside of cell (FIG. 1). Exosome is found in various human body fluids (for example, blood, urine, saliva, amniotic fluid, ascites, pleural effusion, fluid in the glenoid cavity, and human milk), and it is known that there are a membrane protein, an intracellular protein, RNA, DNA, and microRNA in Exosome. In addition, exosome is validated as having high value for being applied for a diagnosis and treatment. Examples of exosome functions that are known include cell death, angiogenesis, inflammation, blood coagulation, interaction between cell and environment, and the like.

### CITED DOCUMENTS

Korean Patent Publication No. 10-2009-0047289

Korean Patent Publication No. 10-2010-0058421

### SUMMARY OF THE INVENTION

The present invention is designed by the above requirements, and an object of the present invention provides novel exosomal proteins.

In order to achieve the above object, the present invention provides an exosomal protein composition including at least one protein selected from the group consisting of proteins having gene information numbers listed in the following Table.

TABLE 1

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |

According to an embodiment of the present invention, it is preferable that the proteins are originated from human, but the present invention is not limited thereto.

Also, the present invention provides a composition for diagnosing an age-related macular degeneration, in which the composition includes at least one protein selected from the group consisting of proteins having gene information numbers listed in the following Table or antibodies against the proteins.

TABLE 2

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |

In addition, the present invention provides a composition for preventing and treating an age-related macular degeneration, in which the composition includes at least one protein selected from the group consisting of proteins having gene information numbers listed in the following Table or antibodies against the proteins.

TABLE 3

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |

In addition, the present invention provides a method for obtaining information on an age-related macular degeneration from a subject, in which the method includes (a) measuring at least one biomarker in a biological sample obtained from a subject, in which at least one biomarker is selected from the composition disclosed in claim 1 or 3 of the present invention; and (b) correlating the sample with an age-related macular degeneration when the measured value or values is/are higher than that of the normal human.

The antibody of the present invention may be a polyclonal antibody, or preferably, monoclonal antibody.

The polyclonal antibody may be prepared by injecting a biomarker protein or fragments thereof as an immunogen to an external host according to the method known by a person skilled in the related art. Examples of the external host include a mammal such as a mouse, a rat, a sheep, and a rabbit. The immunogen is injected intramuscularly, abdominally, or subcutaneously. Generally, the immunogen is injected along with adjuvant to increase antigenicity. Blood is regularly collected from the external host, thereby collecting serum exhibiting improved titer or specificity to an antigen, or isolating and purifying an antibody from the blood.

The monoclonal antibody may be prepared by a technology [Koeher and Milstein 1975, Nature, 256:495)] of generating a cell line immortalized by the fusion that is known by a person skilled in the related art. A brief explanation of the generating method is as follows. First, 20 μg of a pure protein was obtained and then immunized to a Balb/C mouse, or a peptide was synthesized and bound to bovine serum albumin, and then immunized to the mouse. Since then, antigen-producing lymphocyte was isolated from the mouse, and then fused with myeloma of human or a mouse to generate immortalized hybridoma. Using an ELISA method, hybridoma that produces the desired monoclonal antibody was only selected and then proliferated, and then the monoclonal antibody was isolated and purified from the culture.

In order to achieve another purpose of the present invention, there is provided a method of obtaining information about an age-related macular degeneration of a subject, in which the method includes detecting in the presence of the biomarker protein of the present invention or immunogenic fragments thereof from a body fluid of the subject.

In the method of the present invention, using human blood, the detecting in the presence of a biomarker protein or immunogenic fragments thereof from the liver blood solution of a subject is directly performed by using a two-dimensional (2-D) electrophoresis, or the detecting in the presence of a biomarker protein or immunogenic fragments thereof is indirectly performed through an antigen-antibody reaction performed by contacting the blood with the antibody of the present invention.

Examples of an immunoassay method that is now widely known as the antigen-antibody reaction include an enzyme-linked immunosorbent assay (ELISA, Coated tube), a magnetic particle method, in which an antibody-binding magnetic particle is bound to a tube, and then an antigen-tracer and non-degradable contaminant are competitively reacted each other to induce an enzyme reaction, thereby quantifying, and a latex particle method using an antibody-binding latex particle.

In order to achieve still another object of the present invention, there is provided a kit for diagnosing an age-related macular degeneration, in which the kit includes an antibody specifically binding to a biomarker protein of the present invention or immunogenic fragments thereof as an effective component.

The diagnosing kit of the present invention is prepared by the preparing method that is known by a person skilled in the related art, and typically includes an antibody, a buffer, a stabilizer, an inert protein in a freeze-drying type. The antibody may be labeled with radioisotope, fluorogen, enzyme, and the like.

The monoclonal antibody of the present invention may be variously used for an immunoassay kit (ELISA, antibody coated tube test, lateral-flow test, potable biosensor), and may be also used for developing a protein chip having various spectrums for detecting an age-related macular degeneration through a development of an antibody exhibiting higher specificity and sensitivity.

In order to achieve another object of the present invention, there is provided a composition for treating and preventing an age-related macular degeneration, in which the composition includes a protein selected from the group increasing and decreasing an aqueous humor of a patient with an age-related macular degeneration as an effective component.

The pharmaceutical composition of the present invention may be prepared in a formulation, such as parenteral injections by mixing with a pharmaceutically acceptable carrier, excipient, diluents, and the like according to the method that is known in a pharmaceutical field, and then administered intravenously. A dose of the composition according to the present invention may be properly selected according to an age, sex, conditions, and disease symptom of a patient and the dose may be administered in an amount of 0.001 to 100 mg per a day based on an adult standard.

Hereinafter, the present invention will be described.

The present inventors first confirmed the presence of exosome in an aqueous humor of the living human, and also confirmed the presence of total 145 proteins in exosome by analyzing the proteins in the exosome using a proteomic protein technique.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a mimetic diagram illustrating an exosome generation;

FIG. 2 is a mimetic diagram illustrating a method of obtaining exosome using ExoQuick in ARPE-19 cell and an aqueous humor (AH) of a patient with a wet macular degeneration;

FIG. 3 is photographs illustrating a confirmation of exosome obtained from an ARPE-19 cell and an aqueous humor (AH), in which (A) the exosome having a relative uniform size of 100 nm or less was confirmed by using an electron microscope (scale bar, 100 nm), and (B) the presence of CD63 that is an exosome marker was confirmed by using a western blot, in which in order from the left column, there are exosome (con: control culture, para: culture exposed to 400 μM paraquat, 24 h) obtained from cell lysate used for obtaining exosome and obtained from a conditioned medium (CM), and exosome obtained from an aqueous humor of a patient, and it was confirmed that there is exosome in cathepsin D.

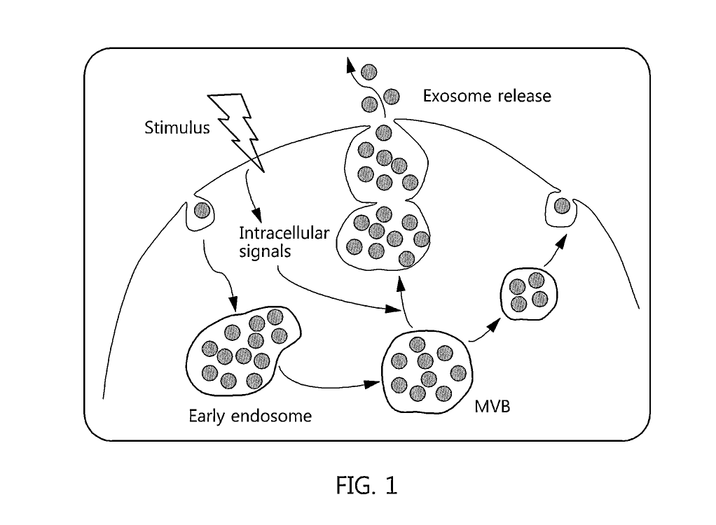

### DETAILED DESCRIPTION OF THE PREFERRED EMBODIMENT

Exemplary embodiments of the present invention will now be described in detail with reference to the accompanying drawings.

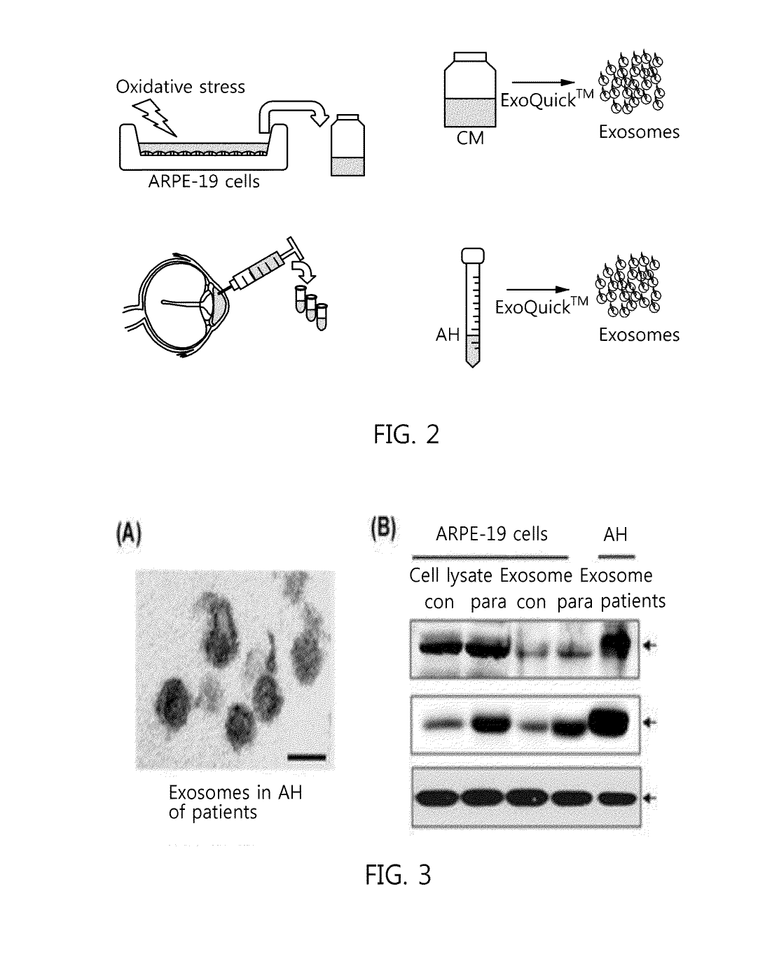

A method of isolating exosome and a method of identifying properties of exosome are as follows. In order to remove dead cells and cell debris, after centrifuging at 3000×g for 15 minutes, the cells were mixed with ExoQuick™ reagent (ExoQuick™ Exosome Precipitation Solution (System Bioscience, SBI)) and then maintained at 4° C. overnight. The ExoQuick/sample mixture was centrifuged at 1500×g for 30 minutes to obtain an exosomal pellet. In order to confirm whether or not the obtaining pellet is an exosomal pellet, the following processes were performed. First, the obtaining pellet was confirmed to be a relative uniform and round vesicle having a diameter of 50 to 100 nm by using an electron microscope, and secondly, it was confirmed there was an exosomal marker protein (CD63) that was well known by using a western blot analysis (FIGS. 2 and 3).

The total 49 novel exosomal proteins were found by comparing 145 proteins with the newest list of ExoCarta (exocarta.org) having the list of the exosomal proteins. It is estimated that the exosome found in an aqueous humor is originated from various cells in eyeball. Compared with exosome extracted from culture solution of retinal pigmented epithelial cell, there is a good chance that the retinal pigmented epithelial cell may be a main cell in eyeball secreting exosome. The exosome secreted by the retinal pigmented epithelial cell has relevance to a mechanism generating drusen (extracellular deposit) that is a risk factor of occurring and progressing an age-related macular degeneration. Accordingly, the exosome may come to the fore as a new cause of an age-related macular degeneration.

TABLE 4

| | | entrez | | |
|---|---|---|---|---|
| No. | Accession # | ID | Protein Name | MW |
| 1 | 207028673 | 1646 | aldo-keto reductase family | 15738.19 |
| | | | 1 member C2 isoform 2 | |
| | | | [Homo sapiens] | |
| 2 | 4502261 | 462 | antithrombin-III precursor | 52568.98 |
| | | | [Homo sapiens] | |
| 3 | 206597441 | 218 | aldehyde dehydrogenase, | 50362.9 |
| | | | dimeric NADP-preferring | |
| | | | [Homo sapiens] | |
| 4 | 4502491 | 708 | complement component 1 Q | 31342.61 |
| | | | subcomponent-binding | |
| | | | protein, mitochondrial | |
| | | | precursor [Homo sapiens] | |
| 5 | 5031777 | 3419 | isocitrate dehydrogenase | 39566.1 |
| | | | [NAD] subunit alpha, | |
| | | | mitochondrial precursor | |
| | | | [Homo sapiens] | |
| 6 | 24234747 | 3608 | interleukin enhancer-binding | 43035.2 |
| | | | factor 2 [Homo sapiens] | |
| 7 | 46094076 | 25987 | tsukushin precursor | 37783.8 |
| | | | [Homo sapiens] | |
| 8 | 4501989 | 174 | alpha-fetoprotein precursor | 68633.09 |
| | | | [Homo sapiens] | |
| 9 | 260064050 | 5345 | alpha-2-antiplasmin | 47876.72 |
| | | | isoform b precursor | |
| | | | [Homo sapiens] | |
| 10 | 4503519 | 8665 | eukaryotic translation | 37540.17 |
| | | | initiation factor 3 subunit | |
| | | | F [Homo sapiens] | |
| 11 | 217272892 | 9343 | 116 kDa U5 small nuclear | 109366.4 |
| | | | ribonucleoprotein component | |
| | | | isoform a [Homo sapiens] | |
| 12 | 14149738 | 57486 | neurolysin, mitochondrial | 80599.83 |
| | | | precursor [Homo sapiens] | |
| 13 | 15451921 | 80310 | platelet-derived growth | 42139.92 |
| | | | factor D isoform 2 | |
| | | | precursor [Homo sapiens] | |
| 14 | 4885649 | 10054 | SUMO-activating enzyme | 71179.34 |
| | | | subunit 2 | |
| | | | [Homo sapiens] | |
| 15 | 290560750 | 26762 | hepatitis A virus cellular | 39224.98 |
| | | | receptor 1 precursor | |
| | | | [Homo sapiens] | |
| 16 | 32483374 | 10528 | nucleolar protein 56 | 66008.77 |
| | | | [Homo sapiens] | |
| 17 | 7305503 | 30968 | stomatin-like protein 2 | 38510.22 |
| | | | [Homo sapiens] | |
| 18 | 71773106 | 163 | AP-2 complex subunit beta | 105624.8 |
| | | | isoform a [Homo sapiens] | |
| 19 | 169218225 | 652147 | PREDICTED: u5 small | 194198.9 |
| | | | nuclear ribonucleoprotein | |
| | | | 200 kDa helicase-like, | |
| | | | partial [Homo sapiens] | |
| 20 | 45446745 | 1646 | aldo-keto reductase family | 36711.99 |
| | | | 1 member C2 isoform | |
| | | | 1 [Homo sapiens] | |
| 21 | 24497583 | 8644 | aldo-keto reductase family 1 | 36829.86 |
| | | | member C3 [Homo sapiens] | |
| 22 | 14141161 | 3192 | heterogeneous nuclear | 88924.16 |
| | | | ribonucleoprotein | |
| | | | U isoform b | |
| | | | [Homo sapiens] | |
| 23 | 4885377 | 3007 | histone H1.3 | 22336.33 |
| | | | [Homo sapiens] | |
| 24 | 291291012 | 441282 | putative aldo-keto reductase | 39435.3 |
| | | | family 1 member B15 | |
| | | | [Homo sapiens] | |
| 25 | 205277471 | 1690 | cochlin precursor | 59445.33 |
| | | | [Homo sapiens] | |
| 26 | 258613957 | 444 | aspartyl/asparaginyl beta- | 21949.51 |
| | | | hydroxylase isoform 1 | |
| | | | [Homo sapiens] | |
| 27 | 4505047 | 4060 | lumican precursor | 38404.8 |
| | | | [Homo sapiens] | |
| 28 | 4503635 | 2147 | prothrombin preproprotein | 69992.2 |
| | | | [Homo sapiens] | |
| 29 | 15890086 | 1287 | collagen alpha-5(IV) chain | 161530.3 |
| | | | isoform 2 precursor | |
| | | | [Homo sapiens] | |
| 30 | 47578117 | 80243 | phosphatidylinositol 3,4,5- | 112019 |
| | | | trisphosphate-dependent Rac | |
| | | | exchanger 2 protein isoform | |
| | | | b [Homo sapiens] | |
| 31 | 290656011 | 4756 | neogenin isoform 2 precursor | 154206.7 |
| | | | [Homo sapiens] | |
| 32 | 5454158 | 7407 | valyl-tRNA synthetase | 140387.4 |
| | | | [Homo sapiens] | |
| 33 | 20544168 | 3010 | histone H1t | 22005.67 |
| | | | [Homo sapiens] | |
| 34 | 22035638 | 4257 | microsomal glutathione | 17587.22 |
| | | | S-transferase 1 | |
| | | | [Homo sapiens] | |
| 35 | 55770836 | 51097 | probable saccharopine | 47121.48 |
| | | | dehydrogenase | |
| | | | [Homo sapiens] | |
| 36 | 153945728 | 4131 | microtubule-associated | 270465.9 |
| | | | protein 1B | |
| | | | [Homo sapiens] | |
| 37 | 83376130 | 1933 | elongation factor 1-beta | 24748.28 |
| | | | [Homo sapiens] | |
| 38 | 296317339 | 7417 | voltage-dependent anion- | 31546.54 |
| | | | selective channel protein | |
| | | | 2 isoform 2 | |
| | | | [Homo sapiens] | |
| 39 | 133925811 | 3842 | transportin-1 isoform 1 | 102288.9 |
| | | | [Homo sapiens] | |
| 40 | 208431836 | 8761 | polyadenylate-binding | 69534.97 |
| | | | protein 4 isoform 3 | |
| | | | [Homo sapiens] | |
| 41 | 28373105 | 489 | sarcoplasmic/endoplasmic | 114857.2 |
| | | | reticulum calcium | |
| | | | ATPase 3 isoform | |
| | | | e [Homo sapiens] | |
| 42 | 15431301 | 6129 | 60S ribosomal protein | 29207.21 |
| | | | L7 [Homo sapiens] | |
| 43 | 46593007 | 7384 | cytochrome b-c1 complex | 52612.5 |
| | | | subunit 1, mitochondrial | |
| | | | precursor [Homo sapiens] | |
| 44 | 38327552 | 10146 | ras GTPase-activating | 52132.14 |
| | | | protein-binding protein | |
| | | | 1 [Homo sapiens] | |
| 45 | 21396489 | 9361 | lon protease homolog, | 106422.5 |
| | | | mitochondrial precursor | |
| | | | [Homo sapiens] | |
| 46 | 148277071 | 7296 | thioredoxin reductase 1, | 70711.41 |
| | | | cytoplasmic isoform 3 | |
| | | | [Homo sapiens] | |
| 47 | 148596949 | 9221 | nucleolar and coiled- | 73559.65 |
| | | | body phosphoprotein | |
| | | | 1 [Homo sapiens] | |
| 48 | 4504425 | 3146 | high mobility group | 24878.18 |
| | | | protein B1 | |
| | | | [Homo sapiens] | |
| 49 | 148491070 | 1503 | CTP synthase 1 | 66648.09 |
| | | | [Homo sapiens] | |

Table 4 is a list of novel exosomal proteins derived from an aqueous humor of a patient screened in the present invention.

As set forth above, according to exemplary embodiments of the invention, total 49 novel exosomal proteins are found, and it is estimated that the exosome found in an aqueous humor is originated from various cells in eyeball. Compared with exosome extracted from culture solution of retinal pigmented epithelial cell, there is a good chance that the retinal pigmented epithelial cell may be a main cell in eyeball secreting exosome, and thus the exosome secreted by the retinal pigmented epithelial cell has relevance to a mechanism generating drusen (extracellular deposit) that is a risk factor of occurring and progressing an age-related macular degeneration. Accordingly, the exosome may come to the fore as a new cause of an age-related macular degeneration.

While the present invention has been shown and described in connection with the exemplary embodiments, it will be apparent to those skilled in the art that modifications and variations can be made without departing from the spirit and scope of the invention as defined by the appended claims.

What is claimed is:

1. An exosomal protein composition comprising proteins having gene information numbers listed in the following Table:

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |

2. The exosomal protein composition of claim 1, wherein the protein is derived from human.

3. A composition for diagnosing an age-related macular degeneration, the composition comprising at least one protein selected from the group consisting of proteins having gene information numbers listed in the following Table or antibodies against the proteins:

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor |
| | | [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial |
| | | [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |

4. A composition for diagnosing an age-related macular degeneration, the composition comprising proteins having gene information numbers listed in the following Table or antibodies against the proteins:

| No. | Accession # | Protein Name |
|---|---|---|
| 1 | 207028673 | aldo-keto reductase family 1 member C2 |
| | | isoform 2 [Homo sapiens] |
| 2 | 4502261 | antithrombin-III precursor [Homo sapiens] |
| 3 | 206597441 | aldehyde dehydrogenase, dimeric NADP-preferring |
| | | [Homo sapiens] |
| 4 | 4502491 | complement component 1 Q subcomponent-binding |
| | | protein, mitochondrial precursor |
| | | [Homo sapiens] |
| 5 | 5031777 | isocitrate dehydrogenase [NAD] subunit alpha, |
| | | mitochondrial precursor [Homo sapiens] |
| 6 | 24234747 | interleukin enhancer-binding factor 2 |
| | | [Homo sapiens] |
| 7 | 46094076 | tsukushin precursor [Homo sapiens] |
| 8 | 4501989 | alpha-fetoprotein precursor [Homo sapiens] |
| 9 | 260064050 | alpha-2-antiplasmin isoform b precursor |
| | | [Homo sapiens] |
| 10 | 4503519 | eukaryotic translation initiation factor 3 |
| | | subunit F [Homo sapiens] |
| 11 | 217272892 | 116 kDa U5 small nuclear ribonucleoprotein |
| | | component isoform a [Homo sapiens] |
| 12 | 14149738 | neurolysin, mitochondrial precursor |
| | | [Homo sapiens] |
| 13 | 15451921 | platelet-derived growth factor D isoform 2 |
| | | precursor [Homo sapiens] |
| 14 | 4885649 | SUMO-activating enzyme subunit 2 |
| | | [Homo sapiens] |
| 15 | 290560750 | hepatitis A virus cellular receptor 1 precursor |
| | | [Homo sapiens] |
| 16 | 32483374 | nucleolar protein 56 [Homo sapiens] |
| 17 | 7305503 | stomatin-like protein 2 [Homo sapiens] |
| 18 | 71773106 | AP-2 complex subunit beta isoform a |
| | | [Homo sapiens] |
| 19 | 169218225 | PREDICTED: u5 small nuclear ribonucleoprotein |
| | | 200 kDa helicase-like, partial |
| | | [Homo sapiens] |
| 20 | 45446745 | aldo-keto reductase family 1 member C2 |
| | | isoform 1 [Homo sapiens] |
| 21 | 24497583 | aldo-keto reductase family 1 member C3 |
| | | [Homo sapiens] |
| 22 | 14141161 | heterogeneous nuclear ribonucleoprotein U |
| | | isoform b [Homo sapiens] |
| 23 | 4885377 | histone H1.3 [Homo sapiens] |
| 24 | 291291012 | putative aldo-keto reductase family 1 |
| | | member B15 [Homo sapiens] |
| 25 | 205277471 | cochlin precursor [Homo sapiens] |
| 26 | 258613957 | aspartyl/asparaginyl beta-hydroxylase |
| | | isoform 1 [Homo sapiens] |
| 27 | 4505047 | lumican precursor [Homo sapiens] |
| 28 | 4503635 | prothrombin preproprotein [Homo sapiens] |
| 29 | 15890086 | collagen alpha-5(IV) chain isoform 2 |
| | | precursor [Homo sapiens] |
| 30 | 47578117 | phosphatidylinositol 3,4,5-trisphosphate- |
| | | dependent Rac exchanger 2 protein |
| | | isoform b [Homo sapiens] |
| 31 | 290656011 | neogenin isoform 2 precursor [Homo sapiens] |
| 32 | 5454158 | valyl-tRNA synthetase [Homo sapiens] |
| 33 | 20544168 | histone H1t [Homo sapiens] |
| 34 | 22035638 | microsomal glutathione S-transferase 1 |
| | | [Homo sapiens] |
| 35 | 55770836 | probable saccharopine dehydrogenase |
| | | [Homo sapiens] |
| 36 | 153945728 | microtubule-associated protein 1B |
| | | [Homo sapiens] |
| 37 | 83376130 | elongation factor 1-beta [Homo sapiens] |
| 38 | 296317339 | voltage-dependent anion-selective channel |
| | | protein 2 isoform 2 [Homo sapiens] |
| 39 | 133925811 | transportin-1 isoform 1 [Homo sapiens] |
| 40 | 208431836 | polyadenylate-binding protein 4 isoform 3 |
| | | [Homo sapiens] |
| 41 | 28373105 | sarcoplasmic/endoplasmic reticulum calcium |
| | | ATPase 3 isoform e [Homo sapiens] |
| 42 | 15431301 | 60S ribosomal protein L7 [Homo sapiens] |
| 43 | 46593007 | cytochrome b-c1 complex subunit 1, mitochondrial |
| | | precursor [Homo sapiens] |
| 44 | 38327552 | ras GTPase-activating protein-binding |
| | | protein 1 [Homo sapiens] |
| 45 | 21396489 | lon protease homolog, mitochondrial |
| | | precursor [Homo sapiens] |
| 46 | 148277071 | thioredoxin reductase 1, cytoplasmic |
| | | isoform 3 [Homo sapiens] |
| 47 | 148596949 | nucleolar and coiled-body phosphoprotein 1 |
| | | [Homo sapiens] |
| 48 | 4504425 | high mobility group protein B1 |
| | | [Homo sapiens] |
| 49 | 148491070 | CTP synthase 1 [Homo sapiens] |
